(19) United States
(12) Patent Application Publication
(10) Pub. No.: US 2025/0269107 A1
(43) Pub. Date: Aug. 28, 2025

(54) System for Infusion of Hazardous Drugs

(71) Applicant: Becton, Dickinson and Company, Franklin Lakes, NJ (US)

(72) Inventors: Zhee Min Jimmy Yong, Singapore (SG); Mum Pew Ng, Singapore (SG); Zi Jun Kwok, Singapore (SG); Ramon Alaiz Chueca, Madrid (ES); Domingo Jesus Ortega Asenjo, El Vellon (ES); Israel Duran, El Molar (ES); Ramdass Palaniswamy, Singapore (SG)

(21) Appl. No.: 18/584,035

(22) Filed: Feb. 22, 2024

(51) Int. Cl. A61M 5/14; A61M 5/142; A61M 39/10

(52) U.S. Cl. CPC A61M 5/1407; A61M 5/1424; A61M 39/10; A61M 2039/1077; A61M 2039/1088

(57) ABSTRACT

An infusion system includes an infusion adapter having a spike configured to pierce a seal of an infusion container, a grip portion extending radially outward relative to the spike, and a connection interface having a membrane, with the infusion adapter defining a passageway extending from a first end of the infusion adapter to the second end of the infusion adapter and the membrane sealing the passageway at the second end of the infusion adapter, and a connection adapter having a spike port and a connector body including a luer connector.

### BACKGROUND OF THE INVENTION

### Field of the Disclosure

The present disclosure relates generally to a system for the infusion of hazardous drugs, including an infusion adapter and a connection adapter.

### Description of the Related Art

Reconstituting, transporting, and administering hazardous drugs, such as cancer treatments, can put health care providers at risk of exposure to these medications and present a major hazard in the health care environment. For example, nurses treating cancer patients risk being exposed to chemotherapy drugs and their toxic effects when administering these potentially harmful drugs due to insecure connections or inadvertent disconnection of the delivery devices or adapters used to transport or administer such drugs. Unintentional chemotherapy exposure can affect the nervous system, impair the reproductive system, and bring an increased risk of developing blood cancers in the future. In order to reduce the risk of health care providers being exposed to toxic drugs, the secure, closed transfer of these drugs is important.

### SUMMARY OF THE INVENTION

In one aspect or embodiment, an infusion system includes an infusion adapter having a spike configured to pierce a seal of an infusion container, a grip portion extending radially outward relative to the spike, and a connection interface having a membrane, with the infusion adapter defining a passageway extending from a first end of the infusion adapter to the second end of the infusion adapter and the membrane sealing the passageway at the second end of the infusion adapter. The system further includes a connection adapter having a spike port and a connector body comprising a luer connector.

The spike port may be connected to the connector body via an interference fit. The luer connector of the connector body may include a male luer lock connector. The grip portion may be annular. The grip portion may be bowl-shaped. A maximum diameter of the grip portion may be 17 mm. The grip portion may include first and second planar surfaces. The grip portion may include first and second openings.

The system may further include a syringe adapter configured to receive the connection interface of the infusion adapter and configured to be connected to the connection adapter via the luer connector. The connection interface may further include a plurality of cantilever arms each having a protrusion.

In a further aspect or embodiment, a method of using the infusion system described above includes: providing a plurality of infusion containers; inserting the spike of the infusion adapter within a port of a first infusion container of the plurality of infusion containers; connecting the connection adapter to the syringe adapter via the luer connector; and connecting the syringe adapter to the infusion adapter via the connection interface.

The method may further include placing the spike port of the connection adapter in fluid communication with a patient infusion line. The method may further include activating an infusion pump. Each of the plurality of infusion containers may include a flexible intravenous bag.

The method may further include: disconnecting the first infusion container from the syringe adapter; inserting a spike of a second infusion adapter within a portion of a second infusion container of the plurality of infusion containers; and connecting the syringe adapter to the second infusion adapter via the connection interface.

### BRIEF DESCRIPTION OF THE DRAWINGS

A more complete understanding of the present disclosure, and the attendant advantages and features thereof, will be more readily understood by reference to the following detailed description when considered in conjunction with the accompanying drawings, wherein:

FIG. 1 is a schematic view of a conventional primary infusion arrangement;

FIG. 2 is a schematic view of a conventional secondary infusion arrangement;

FIG. 3 is a schematic view of a conventional multiway set infusion arrangement;

FIG. 4 is a perspective view of an infusion system according to one aspect or embodiment of the present application;

FIG. 5 is perspective view of a conventional syringe adapter;

FIG. 6 is a perspective view of an infusion adapter of the system of FIG. 4 according to one aspect or embodiment of the present application;

FIG. 7 is a front view of a connection adapter of the system of FIG. 4 according to one aspect or embodiment of the present application;

FIG. 8 is an exploded view of the connection adapter of FIG. 7;

FIG. 9 is a partial cross-sectional view of the connection adapter of FIG. 7; and

FIG. 10 is a schematic view of the infusion system of FIG. 4, showing the infusion system in use.

### DETAILED DESCRIPTION OF THE INVENTION

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

As used herein, the singular form of “a”, “an”, and “the” include plural referents unless the context clearly dictates otherwise.

Spatial or directional terms, such as “left”, “right”, “inner”, “outer”, “above”, “below”, and the like, relate to the embodiments or aspects as shown in the drawing figures and are not to be considered as limiting as the embodiments or aspects can assume various alternative orientations.

All numbers used in the specification and claims are to be understood as being modified in all instances by the term “about”. By “about” is meant within plus or minus twenty-five percent of the stated value. However, this should not be considered as limiting to any analysis of the values under the doctrine of equivalents.

Unless otherwise indicated, all ranges or ratios disclosed herein are to be understood to encompass the beginning and ending values and any and all subranges or subratios subsumed therein. For example, a stated range or ratio of “1 to 10” should be considered to include any and all subranges or subratios between (and inclusive of) the minimum value of 1 and the maximum value of 10; that is, all subranges or subratios beginning with a minimum value of 1 or more and ending with a maximum value of 10 or less. The ranges and/or ratios disclosed herein represent the average values over the specified range and/or ratio.

The terms “first”, “second”, and the like are not intended to refer to any particular order or chronology, but refer to different conditions, properties, or elements.

All documents referred to herein are “incorporated by reference” in their entirety.

The term “at least” is synonymous with “greater than or equal to.”

As used herein, “at least one of” is synonymous with “one or more of.” For example, the phrase “at least one of A, B, or C” means any one of A, B, or C, or any combination of any two or more of A, B, or C. For example, “at least one of A, B, and C” includes A alone; or B alone; or C alone; or A and B; or A and C; or B and C; or all of A, B, and C.

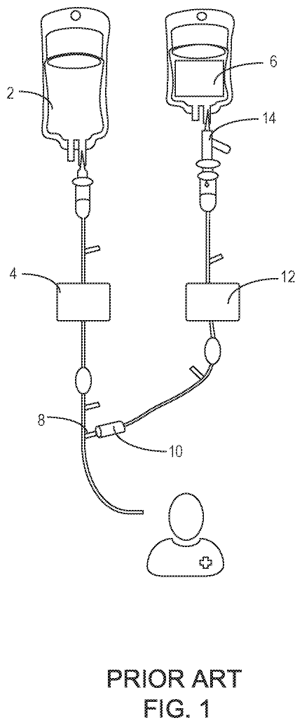

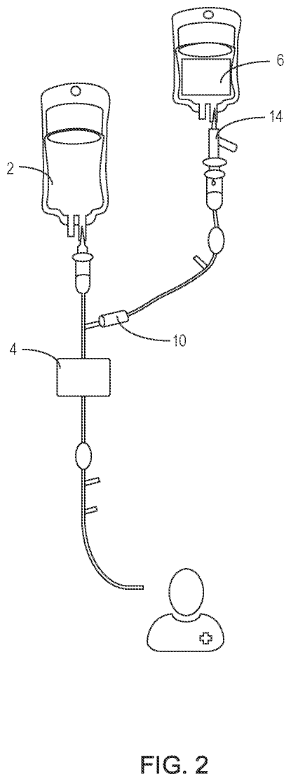

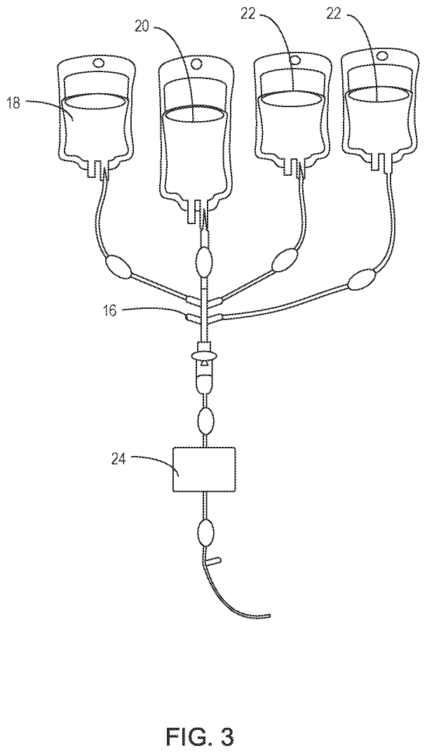

Referring to FIGS. 1-3, conventional infusion arrangements are shown, including a primary infusion arrangement (FIG. 1), a secondary infusion arrangement (FIG. 2), and a multiway set infusion arrangement (FIG. 3). As shown in FIG. 1, with primary infusion, an intravenous bag 2 with saline is connected to a patient with an infusion pump 4 delivering a predetermined flow rate or volume of saline or other fluid to the patient. For delivery of chemotherapeutic medication, a separate intravenous bag 6 with the chemotherapeutic medication is connected to a port 8 of the patient line via a syringe adapter and patient connector 10, with a second infusion pump 12 delivering a predetermined flow rate, volume, or dose of chemotherapeutic medication to the patient. The separate intravenous bag 6 is accessed via an infusion adapter 14. As shown in FIG. 2, with secondary infusion, rather than providing the second infusion pump 12 and connecting downstream of the first infusion pump 4, the separate intravenous bag 6 is connected upstream of the first infusion pump 4. As shown in FIG. 3, with a multiway set infusion, a multiway adapter 16 having a plurality of ports is connected to a pre-med intravenous bag 18, a saline infusion bag 20, and one or more chemotherapeutic infusion bags 22, with infusion delivery via a single infusion pump 24.

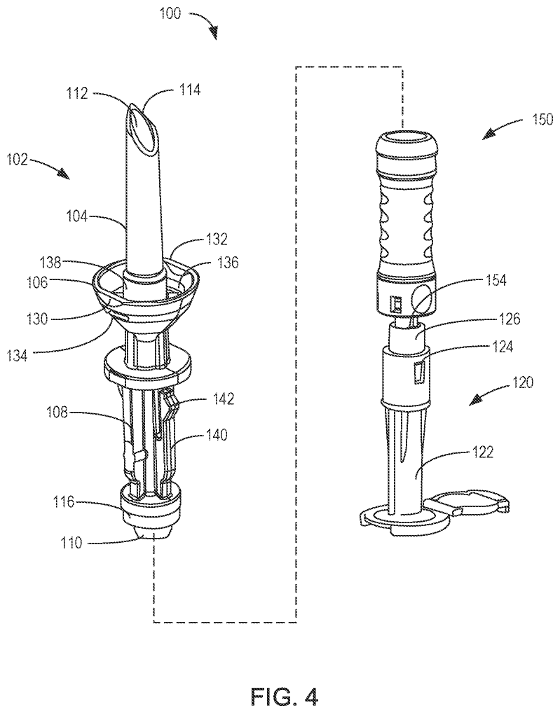

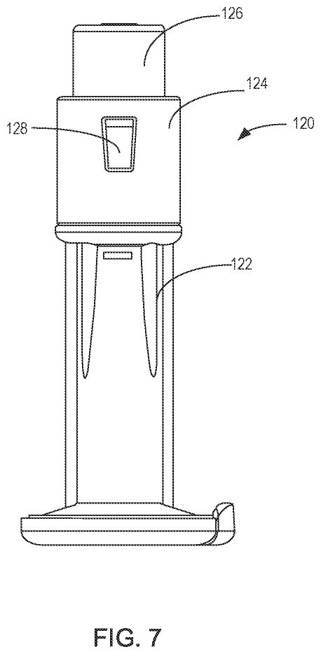

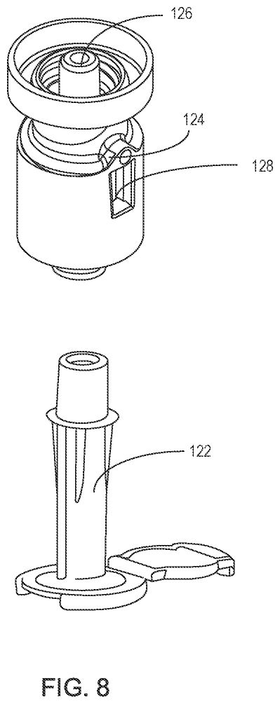

Referring to FIGS. 4-10, in one aspect or embodiment of the present application, an infusion system 100 includes an infusion adapter 102 including a spike 104 configured to pierce a seal of an infusion container, a grip portion 106 extending radially outward relative to the spike 104, and a connection interface 108 including a membrane 110. The infusion adapter 102 defines a passageway 112 extending from a first end 114 of the infusion adapter 102 to the second end 116 of the infusion adapter 102, with the membrane 110 sealing the passageway 112 at the second end 116 of the infusion adapter 102. The infusion system 100 also includes a connection adapter 120 including a spike port 122 and a connector body 124 including a luer connector 126. The infusion system 100 is configured to eliminate priming in pharmacy, decrease priming at bedside, eliminate uncontrolled spiking of intravenous bags, and reducing the number of intravenous lines utilized during infusion. The use of the infusion system 100 is discussed in more detail below.

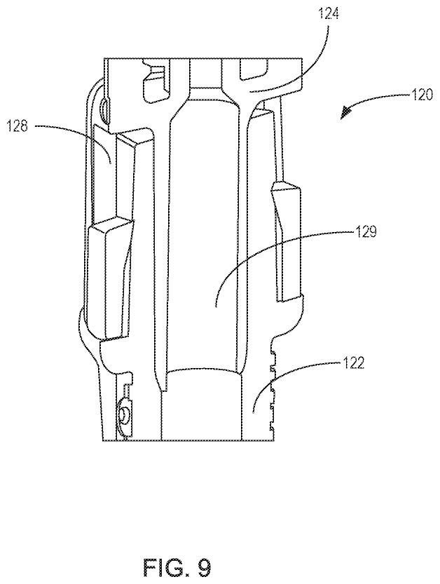

Referring to FIGS. 4, 7, 8, and 9, the spike port 122 is connected to the connector body 124 via an interference fit with interlock features, although other suitable connection arrangements, such as a threaded arrangement, integrally forming the components, a luer connection, or adhering the components, may be utilized. The luer connector 126 of the connector body 124 is shown as a male luer lock connector, although other suitable connection arrangements, such as a threaded arrangement or friction fit connection, may be utilized. The connector body 124 defines at least one opening 128, which is configured to visualize the spike port 122 when the spike port 122 is connected to the connector body 124. An adapter passageway 129 extends through the connection adapter 120, as shown in FIG. 9.

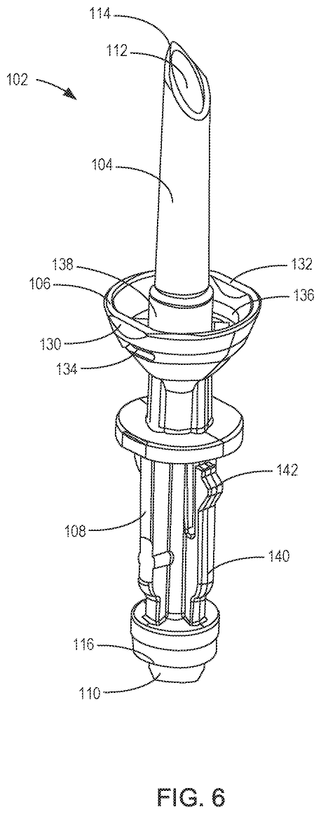

Referring to FIGS. 4 and 6, the grip portion 106 is annular, although other suitable shapes may be utilized. In some aspects or embodiments, the grip portion 106 has a maximum diameter of 17 mm, which reduces the possibility of interfering with other devices when attached to an infusion container and reduces the risk of inadvertent disconnection from the infusion container. In some aspects or embodiments, the grip portion 106 is bowl-shaped, which is configured to maximize the hand comfort of healthcare workers using the infusion adapter 102. The grip portion 106 includes first and second planar surfaces 130, 132. The first and second planar surfaces 130, 132 are configured to be engaged or grasped by a hand of a healthcare worker, and to enable consistent manufacturing assembly that minimizes errors such as misalignment between components. The grip portion 106 includes first and second openings 134, 136. The first and second openings 134, 136 are aligned with the first and second planar surfaces 130, 132 in a longitudinal direction of the infusion adapter 102, although other suitable positions of the first and second planar surfaces 130, 132 may be utilized. The first and second openings 134, 136 are configured to allow visualization of a port of an intravenous bag to ensure the spike 104 of the infusion adapter 102 is sufficiently engaged with the intravenous bag. The spike 104 of the infusion adapter 102 includes an enlarged portion 138 positioned between the first and second ends 114, 116 of the infusion adapter 102. The enlarged portion 138 is configured to secure the infusion adapter 102 to the intravenous bag. The connection interface 108 of the infusion adapter 102 includes a plurality of cantilever arms 140 with a protrusion 142 that are configured to interact and connect to a corresponding connection interface of a mating component.

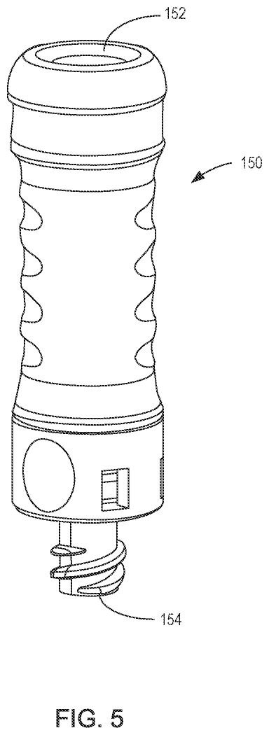

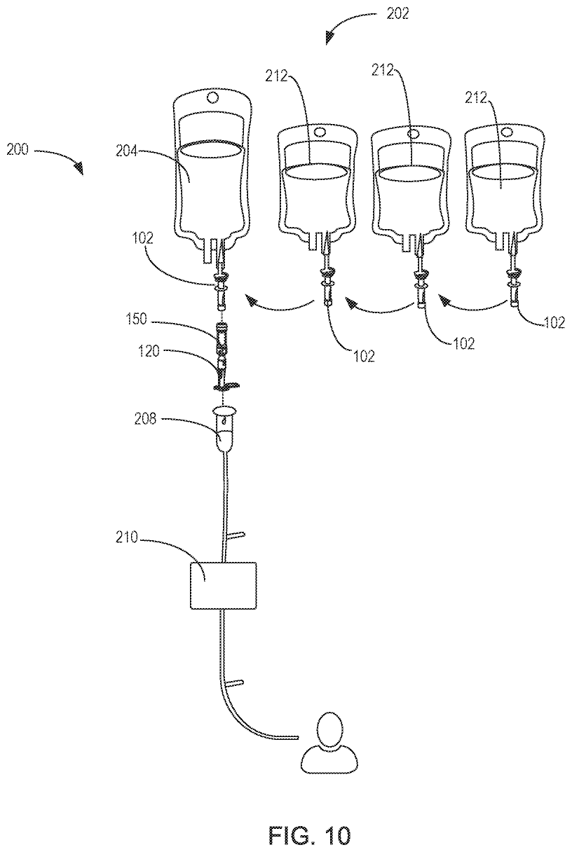

Referring to FIGS. 4 and 10, in some aspects or embodiments, the infusion system 100 includes a syringe adapter 150 configured to receive the connection interface 108 of the infusion adapter 102 and configured to be connected to the connection adapter 120 via the luer connector 126. An example of the syringe adapter 150 is shown in FIG. 5. The syringe adapter 150 is configured to receive at least a portion of the connector interface 108 of the infusion adapter 102 within an opening 152, and is further configured to connect to a syringe (not shown) or other medical device or fluid container via a connector 154, such a female luer connection. The syringe adapter 150 is similar to and operates in a similar manner as the syringe adapter shown and described in U.S. Pat. No. 10,744,315, which is hereby incorporated by reference in its entirety. As shown in FIG. 4, the threads of the connector 152 are covered by the luer connector 126 of the connector body 124 when the syringe adapter 150 is connected to the connector body 124, which provides an indication that the syringe adapter 150 is securely connected to the connector body 124.

Referring to FIG. 10, a method 200 of using the infusion system 100 according to one aspect or embodiment of the present application includes: providing a plurality of infusion containers 202; inserting the spike 104 of the infusion adapter 102 within a port of a first infusion container 204 of the plurality of infusion containers 202; connecting the connection adapter 120 to the syringe adapter 150 via the luer connector 126; and connecting the syringe adapter 150 to the infusion adapter 102 via the connection interface 108.

Referring again to FIG. 10, the method 200 may also include placing the spike port 122 of the connection adapter 120 in fluid communication with a patient infusion line 206. The patient infusion line 206 may include a drip chamber 208 and an infusion pump 210. The method 200 may include activating the infusion pump 210 to deliver a predetermined flow rate or volume of medication or fluid from one of the plurality of infusion containers 202. As shown in FIG. 9, each of the plurality of infusion containers 202 is a flexible intravenous bag, although other suitable containers may be utilized.

Referring again to FIG. 10, in some aspects or embodiments, the method 200 further includes: disconnecting the first infusion container 204 from the syringe adapter 150; inserting a spike 104 of a second infusion adapter 102 within a portion of a second infusion container 212 of the plurality of infusion containers 202; and connecting the syringe adapter 150 to the second infusion adapter 102 via the connection interface 108. The first infusion container 204 may include saline and the second infusion container 212 may include pre-meds or chemotherapeutic medication.

In some aspect or embodiments, the spike 104 of the infusion adapter 102 may be connected to one of the plurality of infusion containers 202 in a controlled environment, fume hood, and/or biosafety cabinet. The connection adapter 120 can be threaded onto the connector 154 of the syringe adapter 150 with the spike port 122 of the connection adapter 120 connected to the patient infusion line 206, with the syringe adapter 150 connected to the connection interface 108 of the infusion adapter 102 to place contents of one of the infusion containers 202 in fluid communication with the patient infusion line 206. The connection between the syringe adapter 150 and the connection interface 108 of the infusion adapter 102 ensures a closed transfer of fluid and prevents leakage or discharge of chemotherapeutic fluids from the infusion system 100.

It will be appreciated by persons skilled in the art that the present disclosure is not limited to what has been particularly shown and described herein above. In addition, unless mention was made above to the contrary, it should be noted that all of the accompanying drawings are not to scale. Of note, the system components have been represented where appropriate by conventional symbols in the drawings, showing only those specific details that are pertinent to understanding the embodiments of the present disclosure, so as not to obscure the disclosure with details that will be readily apparent to those of ordinary skill in the art having the benefit of the description herein. Moreover, while certain embodiments or figures described herein may illustrate features not expressly indicated on other figures or embodiments, it is understood that the features and components of the examples disclosed herein are not necessarily exclusive of each other and may be included in a variety of different combinations or configurations without departing from the scope and spirit of the disclosure. A variety of modifications and variations are possible in light of the above teachings without departing from the scope and spirit of the disclosure, which is limited only by the following claims.

1. An infusion system comprising:
an infusion adapter comprising a spike configured to pierce a seal of an infusion container, a grip portion extending radially outward relative to the spike, and a connection interface comprising a membrane, the infusion adapter defining a passageway extending from a first end of the infusion adapter to the second end of the infusion adapter, the membrane sealing the passageway at the second end of the infusion adapter; and
a connection adapter comprising a spike port and a connector body comprising a luer connector.

2. The infusion system of claim 1, wherein the spike port is connected to the connector body via an interference fit.

3. The infusion system of claim 1, wherein the luer connector of the connector body comprises a male luer lock connector.

4. The infusion system of claim 1, wherein the grip portion is annular.

5. The infusion system of claim 1, wherein the grip portion is bowl-shaped.

6. The infusion system of claim 1, wherein a maximum diameter of the grip portion is 17 mm.

7. The infusion system of claim 1, wherein the grip portion comprises first and second planar surfaces.

8. The infusion system of claim 1, wherein the grip portion comprises first and second openings.

9. The infusion system of claim 1, further comprising a syringe adapter configured to receive the connection interface of the infusion adapter and configured to be connected to the connection adapter via the luer connector.

10. The infusion system of claim 1, wherein the connection interface comprises a plurality of cantilever arms each having a protrusion.

11. A method of using an infusion system, the method comprising:
providing a plurality of infusion containers, each of the plurality of infusion containers including a seal;
providing a syringe adapter;
providing an infusion adapter, comprising a spike configured to pierce a seal of the plurality of infusion containers, the infusion adapter also including a grip portion extending radially outward relative to the spike, and a connection interface comprising a membrane, the infusion adapter defining a passageway extending from a first end of the infusion adapter to the second end of the infusion adapter, the membrane sealing the passageway at the second end of the infusion adapter; and a connection adapter comprising a spike port and a connector body comprising a luer connector;
inserting the spike of the infusion adapter within the seal of a first infusion container of the plurality of infusion containers;
connecting the connection adapter to the syringe adapter via the luer connector; and
connecting the syringe adapter to the infusion adapter via the connection interface.

12. The method of claim 11, further comprising:
placing the spike port of the connection adapter in fluid communication with a patient infusion line.

13. The method of claim 12, further comprising:
activating an infusion pump.

14. The method of claim 11, wherein each of the plurality of infusion containers comprises a flexible intravenous bag.

15. The method of claim 11, further comprising:
disconnecting the first infusion container from the syringe adapter;
inserting a spike of a second infusion adapter within a portion of a second infusion container of the plurality of infusion containers; and
connecting the syringe adapter to the second infusion adapter via the connection interface.
